(19) United States
(12) Patent Application Publication
(10) Pub. No.: US 2020/0268940 A1
(43) Pub. Date: Aug. 27, 2020

(54) LIQUID PLATELET-RICH FIBRIN AS A CARRIER SYSTEM FOR BIOMATERIALS AND BIOMOLECULES

(71) Applicants: Richard J. MIRON, Venice, FL (US); Yufeng ZHANG, Wuhan (CN)

(72) Inventors: Richard J. MIRON, Venice, FL (US); Yufeng ZHANG, Wuhan (CN)

(21) Appl. No.: 16/063,697

(22) PCT Filed: May 15, 2017
(86) PCT No.: PCT/US17/32772

§ 371 Date: Jun. 18, 2018

(51) Int. Cl. A61L 27/36; A61L 27/22; A61L 27/54; A61K 38/36; A61K 35/19; A61L 27/24; A61L 27/20; A61L 27/38; B01D 21/26

(52) U.S. Cl. CPC A61L 27/3616; A61L 27/225; A61L 27/54; A61K 38/36; A61K 35/19; A61L 2300/414; A61L 27/20; A61L 27/227; A61L 27/3817; B01D 21/262; A61L 2300/104; A61L 27/24

(57) ABSTRACT

Methods are provided for preparing liquid PRF and using the liquid PRF as a drug-delivery carrier system for other regenerative biomaterials and biomolecules. This carrier system provides more effective regenerative strategies for improved tissue wound healing, treatment, and regeneration in the body by supplying additional autologous growth factors into commonly-utilized biomaterials improving the host-tissue response to such therapies.

### PRIORITY CLAIM

This patent application claims priority to and the benefit of the filing date of Patent Cooperation Treaty (PCT) patent application having International Application No. PCT/US17/32772, filed on May 15, 2017, which is incorporated herein in its entirety.

### FIELD OF THE INVENTION

The present invention relates to a method of preparing an isolated serum fraction of platelet rich fibrin (PRF) in a liquid formulation and using the liquid PRF as a drug-delivery carrier system for other regenerative biomaterials and biomolecules.

### BACKGROUND OF THE INVENTION

Currently, several millions of people receive therapy and surgery for a variety of diseases affecting connective tissues including bone, tendon, ligament, cartilage and skin. For example, bone regenerative procedures, caused by trauma, disease, age or congenital disorders, have seen a steady increase in the number of regenerative procedures performed yearly to replace or regenerate missing bone. Similarly, osteoarthritis (OA) is a common disease that affects over 250 million people and involves chronic joint pain, an inadequate healing response, and progressive deterioration of joints. Most currently used treatments are restricted to pain alleviation by a combination of pharmacological and non-pharmacological approaches, such as viscosupplementation. However, the more challenging task is to regenerate the already damaged joint. Thus, research efforts have been directed towards the development of tissue regenerative strategies using various bioactive molecules and biomaterials.

Autologous blood-derived products, such as platelet-rich plasma (PRP), are critical for tissue repair and regeneration. These products can deliver a collection of supra-physiological doses of autologous bioactive molecules (e.g. growth factors) that have important roles in processes including inflammation, angiogenesis, cell migration, bone healing, soft tissue healing, and metabolism in pathological conditions, such as OA. Since angiogenesis is the focus of initiating any tissue healing and bone regeneration, PRP initiates a more rapid and complete healing process by concentrating platelets and growth factors to defective tissues. PRP has shown to have positive effects in tissue repair, OA, tendinitis, and nerve injury.

Some techniques for repairing tissue ischemic events involve the preparation of an isolated liquid serum fraction of PRF without the addition of an anticoagulant. Specifically, the coagel is separated from the PRF by pressing, squeezing, filtering, and/or centrifuging the coagel to isolate the serum fraction containing the fluid fraction of PRF. The serum fraction contains specific factors, such as adhesion molecules, such as fibronectin and vitronectin, and may be administered directly to an individual or combined with various biomaterials. However, these techniques involve centrifugation at high G-forces for extended periods of time and squeezing/pressing the PRF clot which contains all the cells and growth factors. By pressing or squeezing the resulting fibrin clot/matrix to isolate the fluid fraction of PRF, potentially beneficial cells and growth factors are being destroyed, and the high G-forces tends to displace a number of important regenerative cells near the bottom of collection tubes during the centrifugation process, away from the PRF layer. Furthermore, following squeezing or pressing, the majority of important cells responsible for tissue regeneration remain in the PRF clot, not in the PRF coagel.

Consequently, there is a need for a defined method and composition for preparing liquid PRF with various other regenerative strategies for improved tissue wound healing, treatment, and regeneration in the body.

### SUMMARY

What is provided is a method of preparing liquid PRF and using the liquid PRF as a drug-delivery carrier system for other regenerative biomaterials and biomolecules. This carrier system provides more effective regenerative strategies for improved tissue wound healing, treatment, and regeneration in the body by supplying additional autologous growth factors into commonly-utilized biomaterials improving the host-tissue response to such therapies.

In an exemplary embodiment, the method of preparing an isolated serum fraction of liquid PRF comprises spinning a whole blood sample by centrifugation to separate erythrocytes; separating a sample of liquid PRF from the whole blood sample without the addition of an additive; spinning the sample of liquid PRF instantly by centrifugation carried out at 20 to 950 G for 2 to 8 minutes; and prior to formation of a fibrin matrix of PRF, collecting an isolated serum fraction containing the liquid PRF using an application device.

In an exemplary embodiment, the composition comprises an isolated serum fraction of liquid PRF and at least one regenerative biomaterial, the composition being obtained by a method comprises spinning a whole blood sample by centrifugation to separate erythrocytes; separating a sample of liquid PRF from the whole blood sample without the addition of an additive; spinning the sample of liquid PRF instantly by centrifugation carried out at 20 to 950 G for 2 to 8 minutes; collecting the isolated serum fraction containing the liquid PRF using an application device; and combining the isolated serum fraction containing the liquid PRF with the regenerative biomaterial.

In yet another exemplary embodiment, the method of treating an injured tissue in an individual comprises determining a site of tissue injury in the individual; administering, using an injectable application device, an isolated serum fraction of liquid platelet-rich fibrin (PRF) into and around the site of tissue injury; and administering, using an injectable application device, at least one regenerative biomaterial into and around the site of tissue injury thereafter. In some examples, the regenerative biomaterial is administered into and around the site of tissue injury within 20 minutes of administering the serum fraction of liquid PRF into and around the site of tissue injury. In other examples, the serum fraction of liquid PRF and the regenerative biomaterial are combined in the injectable application device to form a composition prior to administering the composition into and around the site of tissue injury. In this example, the composition is administered to the individual prior to the composition forming a fibrin matrix.

### BRIEF DESCRIPTION OF THE DRAWINGS

Subject matter is particularly pointed out and distinctly claimed in the concluding portion of the specification. Claimed subject matter, however, as to structure, organization and method of operation, together with objects, features, and advantages thereof, may best be understood by reference to the following detailed description if read with the accompanying drawings in which:

FIG. 1 is shows exemplary embodiments of the volume of liquid PRF present in tubes centrifuged for various speeds and times and the collection of the liquid PRF from the tubes using a syringe;

FIG. 2 shows combining liquid PRF with a regenerative biomaterial using the syringe of FIG. 1;

FIG. 3 shows chondrocytes differentiation in vitro at 3 weeks with Alcian Blue staining to demonstrate an improvement when liquid-PRF was utilized as a carrier for various growth factors, biomolecules, and biomaterials;

FIG. 4 shows knee regeneration in rabbits administered liquid-PRF with regenerative biomaterials;

FIG. 5 shows osteoblast differentiation in vitro through alkaline phosphatase expression for cells administered liquid-PRF with regenerative biomaterials;

FIG. 6 shows bone volume measurements in rat femur defects after administration of liquid-PRF with regenerative biomaterials;

FIG. 7 shows collagen synthesis of skin fibroblasts in vitro administered liquid-PRF with regenerative biomaterials; and

FIG. 8 shows the rescue of periodontal bone loss in vivo by administering liquid-PRF with regenerative biomaterials.

### DETAILED DESCRIPTION

In the following detailed description, numerous specific details are set forth in order to provide a thorough understanding of the examples as defined in the claimed subject matter, and as an example of how to make and use the examples described herein. However, it will be understood by those skilled in the art that claimed subject matter is not intended to be limited to such specific details, and may even be practiced without requiring such specific details. In other instances, well-known methods, procedures, and ingredients have not been described in detail so as not to obscure the invention defined by the claimed subject matter.

As used herein, the terms “platelet-rich plasma” and “PRP” are understood to refer to a volume of plasma that has a platelet concentration greater than the peripheral blood concentration suspended in a solution of plasma, which typically range between about 150,000 per microliter and 350,000 per microliter. PRP is centrifuged using anticoagulants and therefore does not form a fibrin clot. As used herein, the terms “platelet-rich fibrin” and “PRF” are understood to refer to a volume of second generation PRP where autologous platelets and leucocytes are present in a fibrin clot/matrix. PRF does not contain anticoagulants and is therefore more biological and entirely autologous.

As used herein, the terms “fibrin matrix” and “fibrin clot” are understood to refer to a blood clot or a coagel mainly consisting of fibrin fibers, which was formed as a result following the coagulation of fractions of whole blood. A “coagel” is understood as the coagulated phase of blood, which is the jelly-like mass resulting from the conversion of fibrinogen to fibrin to form a fibrin matrix or fibrin clot.

As used herein, the terms “liquid PRF,” “liquid-PRF,” “liquid platelet-rich fibrin,” “injectable PRF,” and “i-PRF” are understood to refer to PRF in a fluid form prior to formation of a fibrin matrix or fibrin clot of PRF. In liquid PRF, liquid fibrinogen and thrombin have yet to be converted to a fibrin clot or coagel. Liquid-PRF remains in its liquid state for between 15-30 minutes prior to fibrin formation and during this time, the liquid-PRF may be administered into a subject or individual.

As used herein, the term “isolated” is understood to refer to a fraction of blood, plasma, or serum that has been sufficiently separated from other fractions or blood components with which it would naturally be associated. In particular, a serum fraction is isolated so as to be separated from the PRF coagel.

As used herein, the terms “subject” and “individual” are understood to refer to a warm-blooded mammal, such as a human being. As used herein, the term “injury” is used in the ordinary sense to refer to, without limitation, to any wound, burn, incision, trauma, tissue damage, tissue degeneration, or ischemic event, such as OA, bone necrosis, or bone ischemia. As used herein, the term “treatment” is used to refer to both prophylactic and therapeutic treatment, in particular to treat, regenerate, repair, or augment a tissue at a target site.

In an exemplary embodiment, this invention refers to a method for preparing an isolated serum fraction of liquid-PRF. In this embodiment, the method comprises spinning a whole blood sample in a tube by centrifugation to separate erythrocytes; separating a sample of liquid platelet-rich fibrin (PRF) from the upper layer of the tube without the addition of an additive (e.g., anti-coagulant); spinning the sample of liquid PRF instantly by centrifugation carried out at 20 to 950 G for 2 to 8 minutes; and collecting an isolated serum fraction containing the liquid-PRF using an application device.

In this embodiment, 10 ml of whole blood samples may be obtained in a first sterile tube. Centrifugation takes place using lower centrifugation speeds and times, such as at 60 G for 3 minutes. The liquid-PRF may then be collected by using a sterile syringe to collect the liquid-PRF. It is preferred to separate the liquid-PRF by avoiding exogenous additives that would possibly contaminate the serum preparation of the liquid-PRF.

It was unexpectedly found that spinning the sample of liquid-PRF by centrifugation carried out at lower speeds/G forces (gradually decreasing the G-force from 950 G towards 60 G) and for a shorter time (between about 2 and 8 minutes) resulted in increased growth factor release of PDGF, TGF-β1, EGF and IGF from the fibrin matrix/clot eventually formed in the subject. This resulted in an improvement in cell proliferation, regeneration, and healing of injured tissue, such as osteoarthritic material. In a preferred embodiment, the liquid-PRF is spun at 60 g for 3 minutes. In other embodiments, the centrifugation procedure to produce the liquid-PRF may be carried out at between 70 and 200 G, within about 2 to 8 minutes. A yellow/orange upper layer corresponding to the liquid-PRF may then be separated from the remaining blood materials.

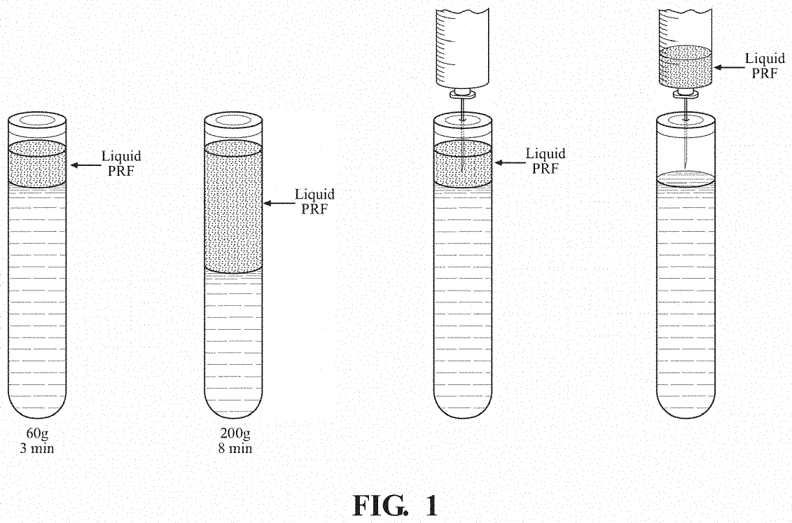

The higher the G-force and time applied to the centrifugation protocol, the higher the volume of liquid-PRF (and larger the layer of liquid-PRF found in the tube). However, this results in a lower concentration of cells and growth factors in the liquid-PRF. Referring to FIG. 1, FIG. 1 shows exemplary embodiments of the volume of liquid PRF present in tubes centrifuged for various speeds and times and the collection of the liquid PRF from the tubes using a syringe. Specifically, centrifugation carried at 60 G for 3 minutes resulted in a smaller volume and layer of liquid-PRF in the tube than centrifugation carried out at 200 G for 8 minutes, but with more density of cells and growth factors. The centrifugation speed and time vary based on the intended use/applications of the liquid-PRF. As shown in FIG. 1, a syringe is used to collect the liquid-PRF from the upper layer of the tube after centrifugation.

The reduction in centrifugation speed to 60 G and time to 3 minutes for collecting a serum fraction of liquid-PRF as compared with currently used techniques, such as those for preparing PRP, prevents the formation of a fibrin matrix/clot. The liquid-PRF (or liquid fibrinogen) is then collected from a tube using an application device, such as a syringe, a needle, or other application device configured to remove the isolated serum fraction of liquid PRF from the upper layer of a tube. In preferred embodiments, the needle is an 18 G hypodermic needle. The liquid-PRF (or fibrinogen) may be collected from the upper layer of the tube after centrifugation. The liquid-PRF (or fibrinogen) is collected without having to press, squeeze, or lyse the sample in any way.

The invention relates to a composition comprising an isolated serum fraction of liquid platelet-rich fibrin (PRF) and at least one regenerative biomaterial. In an exemplary embodiment, the composition is obtained by a method comprising spinning a whole blood sample by centrifugation to separate erythrocytes; separating a sample of liquid platelet-rich fibrin (PRF) from the whole blood sample without the addition of an additive (e.g., anti-coagulant); spinning the sample of liquid PRF instantly by centrifugation carried out at 20 to 950 G for 2 to 8 minutes; collecting an isolated serum fraction containing the liquid-PRF using an application device; and combining the isolated serum fraction containing the liquid-PRF with the regenerative biomaterial in the application device.

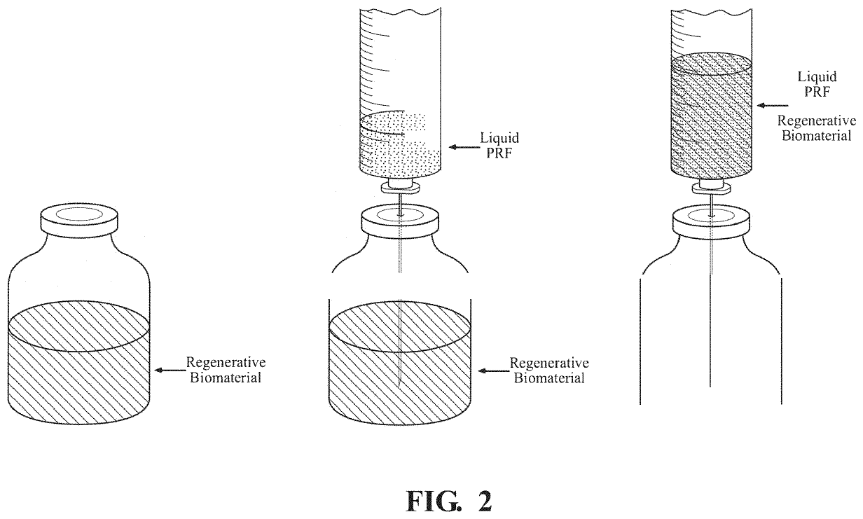

In this embodiment, the isolated serum fraction containing the liquid PRF is collected and combined with the regenerative biomaterial in the application device prior to formation of a fibrin matrix. In order to avoid formation of the fibrin matrix prior to the combination of the liquid-PRF with the regenerative biomaterial, the sample of liquid-PRF is centrifuged in a tube, collected from the tube, and combined with at least one regenerative biomaterial within 15 minutes of being centrifuged. The liquid-PRF (or liquid fibrinogen) is collected from the tube using an application device, such as a syringe or a needle. Referring to FIG. 2, FIG. 2 shows combining liquid PRF with a regenerative biomaterial using the syringe of FIG. 1. Once the liquid PRF and the regenerative biomaterial are combined in the syringe, the combination is ready for injection into an individual.

In some examples, the composition comprises the same amount of the liquid-PRF and the regenerative biomaterial. In some exemplary compositions, the amount of liquid-PRF may be greater than the amount of the regenerative biomaterial, while in other exemplary composition, the amount of the regenerative biomaterial may be greater than the amount of the liquid-PRF.

The regenerative biomaterial may be a growth factor, an injectable material, a cell source, and/or a scaffold material. In some embodiments, the growth factor is selected from the group consisting of BMP1, BMP2, BMP3, BMP4, BMP5, BMP6, BMP7, BMP8, BMP9, BMP10, BMP11, BMP12, BMP13 BMP14, BMP15, BMP16, GDF1, GDF3, GD8, GDF9, GDF12, GDF14, PDGF, IGF, IGF-1, EGF, FGF2, FGF19, amelogenins, enamel matrix proteins, parathyroid hormones, transforming growth factor beta (TGFb1 and 2), epithelial, growth factor (EGF), vascular endothelial growth factor (VEGF) nerve growth factor (NGF), MMPs, TIMPs, caspase inhibitor, B-cell lymphoma 2 (BCL-2), human telemorase reverse transcriptase (hTERT), heat shock protein (HSP) 70, iNOS, IL-1Ra, sTNFR, siRNA, nitric oxide (NO), superoxide anion (02-), IGF-1, IGF-2, bisphosphonates, LIF, COX-1, COX-2, PGE2, PGD2, fibronectin, tenascin, vitronectin, Vitamin E, Vitamin B12, chondroitin sulfates, androgens, thyroid hormones, Stonitum, boron, zinc, magnesium, recombinant growth factors, or any combination thereof.

In these embodiments, the liquid-PRF may be used as a carrier for recombinant growth factors, retroviral vectors, adenoviral vectors, or recombinant adeno-associated viruses for gene therapy purposes. In some examples, the liquid-PRF is used as a carrier for recombinant growth factors and/or gene therapy for (i) inhibiting inflammatory and catabolic pathways; (ii) stimulating anabolic pathways; or (iii) preventing cell senescence and apoptosis. Specifically, the catabolic effect of matrix metalloproteinases (MMPs) may be targeted by the delivery of tissue inhibitors of metalloproteinases (TIMPs), inhibition of tumor growth and vascular endothelial growth factor (VEGF) receptor-2 in vitro, apoptosis, and senescence by inhibitors, such as the caspace inhibitor, B-cell lymphoma 2 (Bcl-2), human telomerase reverse transcriptase (hTERT), heat shock protein (HSP) 70, iNOS, and the like.

Other potential growth factors that may be combined with the liquid-PRF prepared by the method described above include those responsible for increasing the local concentration of the IL-1 receptor antagonist (IL-1Ra); molecules for anti-inflammatory action, such as soluble TNF receptor (sTNFR); specific antibodies/inhibitors (siRNA) for MMPs, aggrecanases, and cathepsins; inflammatory markers; and agents able to decrease reactive oxygen species; molecules for increasing anabolic factors; molecules known to affect intra-signaling pathways of semaphorins, PI3K/AKT, Wnt signaling, or sclerostin, and the like.

In some embodiments, the injectable PRF may be combined with scaffolds, hydrogels, microspheres, Nano fibers, injectable gels, microcarriers, porous injectable particles, collagen, fibrin, silk, HA, alginate, agarose, agar, chitosan, gellan gum, polyethylene glycol (PEG) and its derivatives, poly(Lactide-co-glycolide) (PLGA), poly(l-lactic acid) (PLLA), polyethylene glycol diacrylate (PEGDA), deoxycholic acid, Botox, calcium hydroxylapatite (CaHA), polyacrylamide gel (PAAG), polyalkylimide gel (PAIG), polymethyl methacrylate (PMMA), silicone oil, AFT, injectable containing microparticles of PLLA, carboxymethylcellulose, nonpyrogenic mannitol, magnesium-carboxy-gluconate-hydrolactic gel, liquid injectable silicone, polycaprolactone Microspheres, Poly vinyl alcohol, DHT, dalteparin, and protamine microparticles, or any combination thereof. The scaffolds may include nano-scaffolds, such as those serving to mimic the native extracellular matrix of cartilage, bone, and other tissues in order to assist in their 3-D growth.

In some examples, the porous injectable particles are fabricated via conventional techniques, such as solvent casting, freeze-drying gas foaming, and salt leaching or advanced techniques, such as rapid prototyping and electrospinning. Specifically, the techniques for nanofiber production include electrospinning, self-assembly, and phase separation.

In some embodiments, the scaffold material is selected from the group consisting of titanium, collagen, zirconium, sponges, graft materials, collagen membranes, collagen bi-products comprising synthetic or natural bone material, e-PTFE, d-PTFE, 3-D printing technologies, carbon-based nanomaterials such as carbon nanotubes (CNTs), titanium dioxide (TiO2) nanosheets, graphene, grapheme oxide, synthetic silicates and nanodiamonds (NDs), nHAp-poly(caprolactone) (nHAp-PCL), poly(ester urethane)-urea elastomer (PUEER/PUR), PEUUR nanofibers, synthetic bone grafts, or any combination thereof. In some examples, the graft materials may be derived from autogenous tissues and allografts, such as, demineralized, freeze-dried bone allograft; tissue grafts; xenografts, and the like.

In some embodiments, the cell source is selected from the group consisting of chondrocytes, osteoblasts, mesenchymal stem cells (MSCs), embryonic stem cells (ESCs), skin fibroblasts, fibroblasts mesenchymal stem cells (MSCs), adipocytes, adipose-derived stem cells, or embryonic stem cells (ESCs).

The invention further relates to a method of treating an injured tissue in an individual comprising determining a site of tissue injury in the individual; and administering, using an injectable application device, a composition comprising an isolated serum fraction (liquid PRF) and at least one regenerative biomaterial into and around the site of tissue injury. In this exemplary embodiment, the composition is administered to the individual prior the composition forming a fibrin matrix. As such, the fibrin matrix may form after administration of the composition to the individual.

In another embodiment, the liquid PRF may be administered into and around the site of tissue injury either immediately before or immediately after administration of at least one regenerative biomaterial into or around the same site of tissue injury. In this embodiment, the liquid PRF and the regenerative biomaterial may be mixed inside the tissue of the individual, instead of being mixed in an injectable application device. In order to most effectively treat an individual, the liquid PRF must be administered within a specific predetermined amount of time into the same site of tissue injury where the regenerative biomaterial has already been administered, if the regenerative biomaterial was already administered to the individual. Similarly, the regenerative biomaterial must be administered within a specific predetermined amount of time into the same site of tissue injury where the liquid PRF has already been administered, if the liquid PRF was already administered to the individual. In some embodiments, the predetermined amount of time is 20 minutes.

In this exemplary embodiment, the composition was prepared by the method comprising spinning a whole blood sample by centrifugation to separate erythrocytes; separating a sample of liquid PRF from the whole blood sample without the addition of an additive (e.g., anti-coagulant); spinning the sample of liquid PRF instantly by centrifugation carried out at 20 to 950 G for 2 to 8 minutes; collecting an isolated serum fraction containing the liquid-PRF using an application device; and combining the isolated serum fraction containing the liquid-PRF with the regenerative biomaterial in the application device.

The injured tissue in this exemplary method may be connective tissue, such as bone, tendons, ligaments, cartilage, joint capsules, facial tissues, skin tissues, and the like. Specifically, the types of injured tissues that may be treated using this method include muscle tissue, nerve tissue, vascular tissue, dental tissue, and combinations thereof. In an additional aspect, the exemplary method described herein relates to treating a target site in need of cell proliferation or regeneration, such as skin, a wound, an injury, an incision, or a surgical site. In these aspects, the exemplary method of treatment using the composition comprising liquid-PRF and a regenerative biomaterial (as described herein) promotes wound healing, cell proliferation, and/or regeneration. Thus, this method may be used in regenerative procedures, such as those involving bone, cartilage, facial injections, surgical wounds, or the like.

Further, the administration of the composition disclosed herein is used for treating an individual suffering from OA, osteonecrosis, bone necrosis, tendonitis, impingement syndrome, facial wrinkles, bone grafting, implantation, or the like. For example, the administration of the composition disclosed herein is used to prepare bones or implants, such as dental bone grafts.

The site of administration of the composition disclosed herein is at or near the site of tissue injury and/or damage. The site of tissue injury is determined by established methods, such as those currently utilizing routine injections in the field of osteoarthritis of the knee, facial injections, hair injections, injected within bone grafts for bone augmentations, or combined with barrier membranes for tissue regeneration. Thus, the liquid PRF serves as a drug-delivery carrier system for other regenerative biomaterials and biomolecules.

This invention disclosed herein results in particular advantages, such as increased growth factor release from the liquid-PRF and increased tissue regeneration by increasing fibroblast migration and proliferation, as compared with liquid-PRF centrifuged at or greater than 1000 G for longer than 8 minutes to produce a PRF clot that is then squeezed or lysed to produce a liquid from PRF. Liquid-PRF has many more cells and growth factors released allowing wound healing to take place more effectively when compared to PRP. Specifically, the propagation of bone tissue cells is accelerated and bone tissue regeneration is increased after bone ischemia or any disease which is a consequence of ischemia or related to bone ischemia.

It will, of course, be understood that, although particular examples have been described and are described further below, the claimed subject matter is not limited in scope to a particular example or limitation. Likewise, an example may be implemented in any combination of compositions of matter, apparatuses, methods or products made by a process, for example. The following examples are merely representative of methods of practicing one or more embodiments of the present invention and should not be read as limiting the scope of the invention.

### EXAMPLES

### Example 1

### Use of Liquid-PRF as a Biological Carrier for Hyaluronic Acid, Botox or Collagen/Atelo-Collagen Fillers for Facial Injections

In this study, 1 mL of a 4% concentration of atelo-collagen was mixed with 1 mL of liquid-PRF. Thereafter 1.5 mL of this mixed solution was injected into the naso-labial fold of an individual. An equal strategy was attempted mixing 1 mL of a 24 mg/mL dose of Hyaluronic Acid with 1 mL of liquid-PRF. Once again 1.5 mL of this mixture was injected into the naso-labial fold a patient with satisfactory results. For botulinum toxin, 2 strategies have been attempted. In a first strategy, a 100 unit vial was diluted in 1 mL of saline and then mixed with 1 mL of liquid-PRF. Similarly, a secondary attempt was made whereby a 100 unit vial of botox was diluted in 1 mL of liquid-PRF and utilized for injection thereafter. In this example, liquid PRF is typically centrifuged for 3 minutes at 60 G. However, if more volume is required, liquid PRF may be formulated by spinning for 3-5 minutes at 200 G to produce a larger quantity in mL of liquid PRF in order to have more liquid PRF (carrier) for the other regenerative biomaterials when more liquid is needed to fill facial voids and defects.

### Example 2

### Use of Liquid-PRF as a Carrier for Bone Morphogenetic Protein 2

In the present example, 1.5 mg of rhBMP2 was dissolved in 1 mL of liquid-PRF to reach a final concentration of 1.5 mg/mL (clinically recommended as per standards by Medtronic for the commercially available product Infuse Bone from Medtronic). Thereafter, this combination was mixed with a bone grafting material whereby the liquid-PRF/rhBMP2 was found to form a stable clot around the bone grafting particles. This clot has been shown to degrade over a 14 day period where rhBMP2 may slowly be released over time in a controlled manner and dependent on the rate of degradation of the fibrin clot. The bone grafting material was then utilized in a standardized manner for bone routine bone augmentation within a dental clinic.

### Example 3

### Use of Liquid-PRF as a Carrier for Hyaluronic Acid for Cartilage Regeneration of the Knee

The in vitro and in vivo data obtained support the advantages of using liquid PRF when compared to PRP. In this example, 1 vial of hyaluronic acid Ostenil® (molecular weight of 1.2-1.4×106 Da, 10 mg/mL; TRB Chemedica AG, Munchen, Germany) was mixed with 1 mL of liquid-PRF and shown to drastically improve knee regeneration following injection. These findings highlight the potential for using liquid fibrinogen as a carrier for HA and other potential molecules for osteoarthritis therapy. Similarly, additionally it has been shown that mixing of liquid PRF with a second regenerative material may also take place in vivo. Additionally, 1 mL of liquid PRF may be injected alone and immediately thereafter 1 mL of HA be injected and mixed in vivo. Both strategies have shown to be effective with a preference for pre-mixing ex-vivo within the syringe.

### Example 4

### Use of Liquid-PRF as a Carrier for Silver-Sulfadiazine for Burn/Scar Regeneration

In this example, silver-sulfadiazine (SSD) has been utilized in combination with certain modalities to speed wound healing of burn victims when utilized in combination with various scaffolds. SSD (7.6 mg) is typically dispersed in ethanol (1 mL), the solution (200 L). SSD was then mixed with 1 ml of liquid-PRF and utilized in combination with a collagen scaffold for the treatment of a burn victim.

### Example 5

Use of Liquid-PRF as a Carrier for Regenerative Biomaterials after Chondrocyte Differentiation In Vitro

In this example, chondrocytes were first isolated from rabbit knee cartilage according to standard protocols and incubated at 37° C., under 5% CO₂. Within 12-24 h of incubation, the cells formed an essentially spherical aggregate. For chondrogenic differentiation, cells were induced in chondrogenic induction medium consisting of serum-free DMEM-LG supplemented with ITS+Premix (6.25 μg/ml insulin, 6.25 μg/ml transferrin, 6.25 μg/ml selenious acid, 1.25 mg/ml bovine serum albumin [BSA], 5.35 mg/ml linoleic acid; BD Biosciences, Bedford, Mass.), 10⁻⁷ M dexamethasone (Sigma), 50 μg/ml ascobate-2-phosphate (Sigma) and 10 ng/ml TGF-β1. Medium changes were carried out at 2 to 3-day intervals. During experimental seeding cells were trypsinized and cultured within the following groups discussed below. Following 3 weeks of culture, cells were fixed in 4% formaldehyde and stained for Alcian Blue for chondrogenic differentiation. HA was utilized at a 16 mg/mL concentration.

p(N-isopropylacrylamid-co-butyl methacrylate) (PIB) nanogels were synthesized according to a emulsion polymerization method. Briefly, N-isopropylacrylamide (NIPAM) (2.26 g, 20 mmol), N,N′-methylenebisacrylamide (BIS) (0.03 g, 0.2 mmol), and sodium dodecyl sulfate (SDS) (0.03 g, 0.1 mmol) were dissolved in water (180 mL) for the preparation of the PNIPAM nanogel. For the preparation of the PIB nanogel, butyl methylacrylate (BMA) (0.14 g, 1 mmol) was added to the above solution. The resulting solutions were degassed for at least 60 min by bubbling N2 and heating to 70° C. with stirring. potassium persulfate (KPS) (95 mg, 0.35 mmol) was then added as the polymerization initiator, and the reaction was maintained under an N2 atmosphere at 70° C. for 4.5 h. The resulting PNIPAM and PIB nanogel dispersions were dialyzed (the cutoff molecular weight is 14,000) against water for 2 weeks to remove unreacted monomers and other small molecules. The diameters of the nanogels were determined by dynamic light scattering (DLS). Both nanogels were lyophilized and stored in a desiccator at room temperature. PIB nanogels were used in this study. Furthermore, mesoporous bioactive glass (MBG) containing 5% Strontium have been prepared (Sr-MBG).

Groups included control, 1 mL of PRP, 1 mL of liquid-PRF, 1 mL of 4% atelo-collagen solution, 1 mL of % atelo-collagen with SDF1 (100 ng/ml), 0.5 mL of 4% atelo-collagen+0.5 mL of liquid-PRF (1:1 ratio), 0.5 mL of 4% atelo-collagen+0.5 mL of liquid-PRF+SDF-1 (100 ng/ml), 1 mL of HA (16 mg/mL), 1 mL of HA (16 mg/mL)+SDF-1 (100 ng/ml), 0.5 mL of HA (16 mg/mL)+0.5 mL of liquid-PRF, 0.5 mL of HA (16 mg/mL)+0.5 mL of liquid-PRF+SDF-1 (100 ng/ml), 1 mL of liquid PRF+TIMPs (100 ng/mL), 1 mL of liquid PRF+VEGF (100 ng/mL), 1 mL of liquid PRF+chondroitin sulfate (0.1 mg/mL), 1 mL of PIB nanogel, 0.5 mL of PIB-nanogel+0.5 mL of liquid PRF, 0.5 mL of PIB-nanogel+0.5 mL of liquid-PRF+chondrocytes (2×10⁶ cells), 0.5 mL of PIB-nanogel+0.5 mL of liquid-PRF+BMP9 (100 ng/ml).

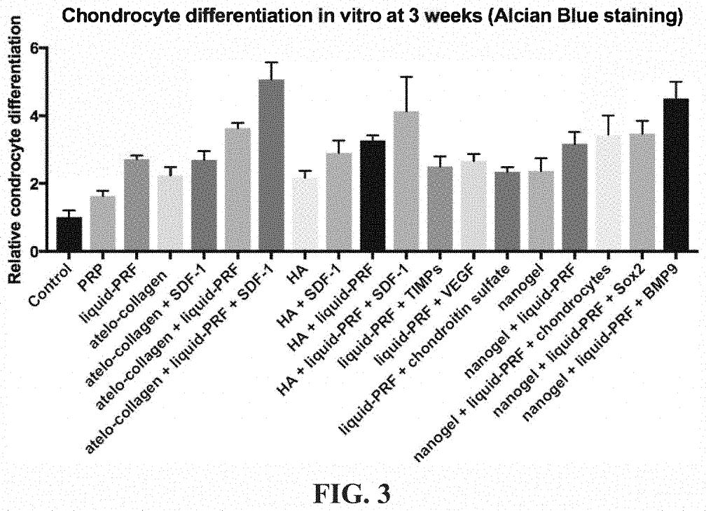

Chondrocytes seeded with each of the groups underwent chondrogenic differentiation for 21 days followed by Alcian-blue staining (FIG. 3). The results demonstrate a marked improvement when liquid-PRF was utilized as a carrier for various biomolecules and biomaterials.

### Example 6

### Use of Liquid-PRF as a Carrier for Regenerative Biomaterials in Knee Regeneration in Rabbits

The present study was carried out on female skeletal mature New Zealand White (NZW) rabbits with a 3 to 4 kg average body weight. All rabbits were given one week to acclimate to the facility prior to surgery and were fed in separate cages in a temperature-controlled room with free access to antibiotic-free food and water.

Rabbits were generally anesthetized intramuscularly using a mixture of 35 mg/kg of ketamine hydrochloride and 5 mg/kg of xylazine. Antibiotic prophylaxis was given 30 min prior to surgery. Following shaving and standard sterile preparation of both lower extremities, the surgical technique was performed bilaterally (right and left knees). The knee joints were opened through a medial para-patellar approach. A 4 cm medial para-patellar arthrotomy was performed and the patella was dislocated laterally to expose the surface of femoro-patellar groove.

Full-thickness defects of 5 mm in diameter and 4 mm in depth were created through the articular cartilage and subchondral bone in the center of the patellar groove in rabbits of each group by using a drill-equipped with a 5 mm drill bit. The defects were then debrided of any remaining osseous or cartilage fragments irrigated with 0.9% sterile saline solution. The defects were filled with various regenerative strategies according to the group allocations. Each wound was closed in layers using simple interrupted sutures (4-0 Vicryl), with final skin closure achieved using a running subcutaneous suturing technique (4-0 Vicryl). Prior to concluding the surgical procedure, the knee was moved through its full range of motion to ensure that normal patellar tracking occurred. All the rabbits were given penicillin (15 mg/kg body weight) for 3 days post-operation.

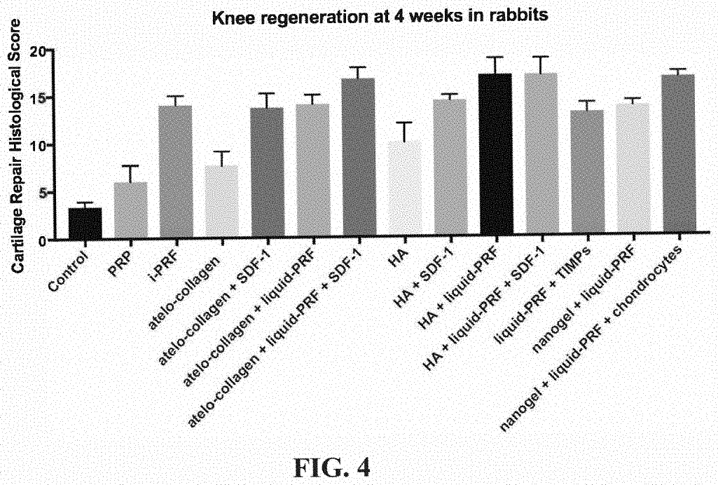

Animals were then sacrificed at 4 weeks post operatively to investigate early wound healing. The samples were examined for gross examination, pCT, and histopathological examination (FIG. 4).

Both knee joints were resected in blocs and placed in 10% formalin for 7 days. Following formalin fixation, the specimens were decalcified in a sodium citrate-formic acid solution for an additional 7 days prior to being embedded in paraffin wax. The specimens were cut into 8 um sections at the posterior, middle, and anterior aspect of the osteochondral site, which provided a representative view of overall lesion healing. The osteochondral sections were stained with hematoxylin and eosin for ICRS histological scoring, alcian blue to assess glycosaminoglycan presence, and processed for type II collagen immunohistochemistry. The ICRS histological score is graded on a scale of 0 to 18 (Table 1).

TABLE 1
International Cartilage Repair Society Histological Score

| Variable | Score |
|---|---|
| Surface | |
| Smooth/continuous | 3 |
| Discontinuities/irregularities | 0 |
| Matrix | |
| Hyaline | 3 |
| Mixture: Cartilage/Fibrocartiolage | 2 |
| Fibrocartilage | 1 |
| Fibrous tissue | 0 |
| Cell Distribution | |
| Columnar | 3 |
| Mixed/columnar clusters | 2 |
| Clusters | 1 |
| Individual cells/disorganized | 0 |
| Cell Population Viability | |
| Predominantly viable | 3 |
| Partially viable | 1 |
| <10% viable | 0 |
| Subchondral Bone | |
| Normal | 3 |
| Increased remodeling | 2 |
| Bone necrosis/granulation tissue | 1 |
| Detached/fracture/callus at base | 0 |
| Cartilage Mineralization | |
| Normal | 3 |
| Abnormal/inappropriate location | 0 |

HA was utilized at a 16 mg/mL concentration and p(N-isopropylacrylamid-co-butyl methacrylate) (PIB) nanogels were synthesized according to a emulsion polymerization method. Briefly, N-isopropylacrylamide (NIPAM) (2.26 g, 20 mmol), N,N′-methylenebisacrylamide (BIS) (0.03 g, 0.2 mmol), and sodium dodecyl sulfate (SDS) (0.03 g, 0.1 mmol) were dissolved in water (180 mL) for the preparation of the PNIPAM nanogel. For the preparation of the PIB nanogel, butyl methylacrylate (BMA) (0.14 g, 1 mmol) was added to the above solution. The resulting solutions were degassed for at least 60 min by bubbling N2 and heating to 70° C. with stirring. potassium persulfate (KPS) (95 mg, 0.35 mmol) was then added as the polymerization initiator, and the reaction was maintained under an N2 atmosphere at 70° C. for 4.5 h. The resulting PNIPAM and PIB nanogel dispersions were dialyzed (the cutoff molecular weight is 14,000) against water for 2 weeks to remove unreacted monomers and other small molecules. The diameters of the nanogels were determined by dynamic light scattering (DLS). Both nanogels were lyophilized and stored in a desiccator at room temperature. PIB nanogels were used in this study. Furthermore, mesoporous bioactive glass (MBG) containing 5% Strontium have been prepared (Sr-MBG).

Groups included control, 1 mL of PRP, 1 mL of liquid-PRF, 1 mL of 4% atelo-collagen solution, 1 mL of % atelo-collagen with SDF1 (100 ng/ml), 0.5 mL of 4% atelo-collagen+0.5 mL of liquid-PRF (1:1 ratio), 0.5 mL of 4% atelo-collagen+0.5 mL of liquid-PRF+SDF-1 (100 ng/ml), 1 mL of HA (16 mg/mL), 1 mL of HA (16 mg/mL)+SDF-1 (100 ng/ml), 0.5 mL of HA (16 mg/mL)+0.5 mL of liquid-PRF, 0.5 mL of HA (16 mg/mL)+0.5 mL of liquid-PRF+SDF-1 (100 ng/ml), 1 mL of liquid PRF+TIMPs (100 ng/mL), 0.5 mL of PIB-nanogel+0.5 mL of liquid PRF, and 0.5 mL of PIB-nanogel+0.5 mL of liquid-PRF+chondrocytes (2×10⁶ cells).

### Example 7

Use of Liquid-PRF as a Carrier for Regenerative Biomaterials with In Vitro Osteoblasts

Primary human osteoblasts were cultured in a humidified atmosphere of 5% CO₂ and 95% relative humidity at 37° C. in growth medium consisting of DMEM (Gibco, Life technologies, Carlsbad, Calif., USA), 10% fetal bovine serum (FBS; Gibco), and 1% Antibiotic/Antimycotic (Gibco). Cells were seeded with the various growth factors, scaffolds, and biomolecules as indicated below within cell culture media at a density of 50,000 cells per well in 24 well dishes for ALP assay. Medium was replaced twice weekly.

Groups included the following: 1 mL of PRP, 1 mL of liquid-PRF, rhBMP2 (100 ng/mL), 1 mL of liquid PRF+rhBMP2 (100 ng/mL), rhBMP9 (100 ng/mL), 1 mL of liquid PRF+rhBMP9 (100 ng/mL), 8 mg of mesoporous bioactive glass (MBG), 1 mL of liquid PRF with 8 mg of mesoporous bioactive glass (MBG), 1 mL of liquid PRF with 8 mg of mesoporous bioactive glass (5% Sr-MBG), 1 mL of HA (16 mg/mL), 0.5 mL of HA (16 mg/mL)+0.5 mL of liquid-PRF, 1 mL of liquid PRF+vit. D, 1 mL of liquid-PRF+alendronate, 1 mL of liquid-PRF+adPDGF, 1 mL of liquid-PRF+siRNA-Sema4d, 1 mL of PIB nanogel, 0.5 mL of PIB-nanogel+0.5 mL of liquid PRF, 0.5 mL of PIB-nanogel+0.5 mL of liquid-PRF+osteoblasts (2×10⁶ cells).

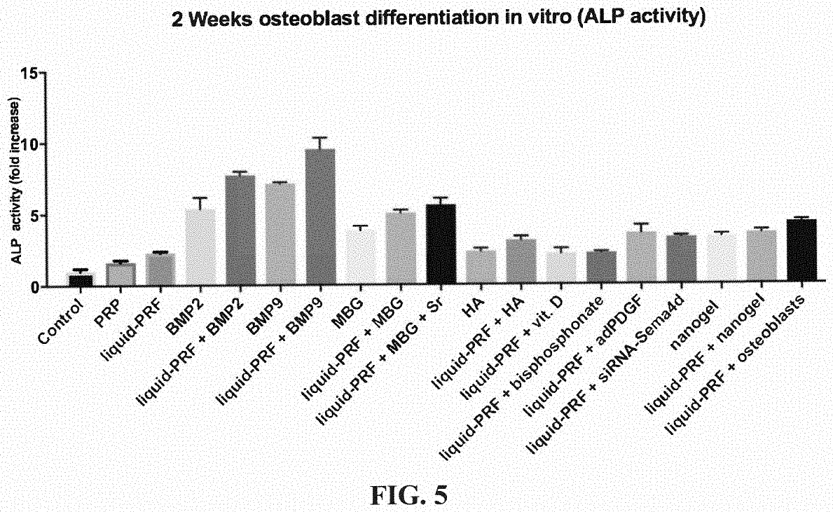

At 14 days, osteoblasts were quantified for alkaline phosphatase expression as determined cell imaging (FIG. 5). Alkaline phosphatase activity was monitored using Leukocyte alkaline phosphatase kit (procedure No. 86, Sigma, St. Louis, Mo., USA). Cells were fixed by immersing in a citrate-acetone-formaldehyde fixative solution for 5 min and rinsed in deionized water for 1 min. Alkaline dye mixture are prepared according to the manufacture's protocol. Surfaces were then placed in alkaline dye mixture solution for 15 min protected from light. After rinsing in deionized water, all images were captured on a Wild Heerbrugg M400 ZOOM Makroskop (WILD HEERBRUGG, Heerbrugg, Switzerland) at the same magnification and light intensity and imported into Image J software (NIH, Bethesda, Md.). Thresholding was used to generate percent stained values for each field of view.

### Example 8

### Use of Liquid-PRF as a Carrier for Regenerative Biomaterials for Bone Volume/Total Volume in Rat Femur Defect

Wistar rats (8 weeks of age, 180-200-g weight) were used in this experiment. The animals were housed in individual wire cages in a temperature- and humidity-controlled room (20-25° C. and 60±5% relative humidity) with a 12-h light/dark cycle and allowed food and water ad libitum. All animals were allowed to acclimate with the laboratory environment for 1 week before surgical procedures were carried out. All operations were carried out under sterile conditions with gentle surgical techniques. The animals were administered intramuscular injections of penicillin (NCPC; 400,000 IU/ml, 0.1 ml/kg day) at the time of surgery and once daily for the next 3 days. No significant pre-operation or post-operation fractures were produced.

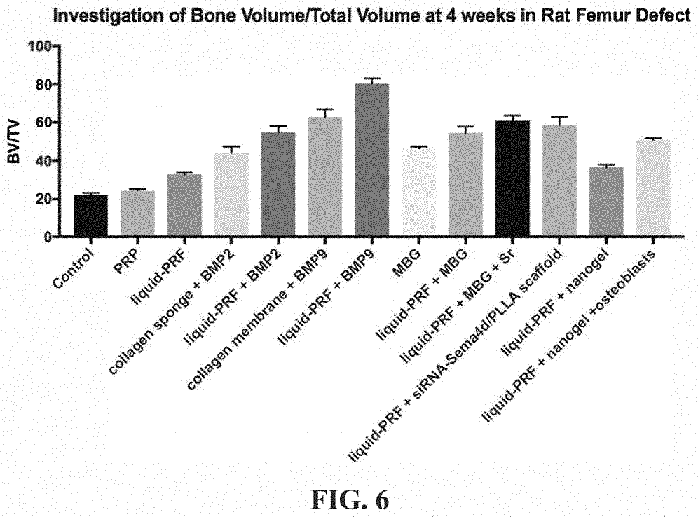

Femur defect drilling was performed under general anesthesia by intraperitoneal injection of sodium pentobarbital (Merck, Darmstadt, Germany; 40 mg/kg body weight). A linear skin incision of approximately 1 cm in the distal femoral epiphysis was made bilaterally, and blunt dissection of the muscles was performed to expose the femoral condyle. Then, a 2.2-mm-diameter anteroposterior bicortical channel was created perpendicular to the shaft axis to remove cancellous bone, by using a trephine bur at a slow speed irrigated under saline solution to avoid thermal necrosis. The drilled holes were rinsed with saline solution in order to remove bone fragments from the cavity. Then each defect was treated with the regenerative modalities and allowed to heal for a 4 weeks (FIG. 6).

Groups included the following: 1 mL of PRP, 1 mL of liquid-PRF, absorbable collagen sponge (ACS)+rhBMP2 (5 ug/mL), 1 mL of liquid PRF+rhBMP2 (5 ug/mL), ACS+rhBMP9 (5 ug/mL), 1 mL of liquid PRF+rhBMP9 (5 ug/mL), mesoporous bioactive glass (MBG), 1 mL of liquid PRF with mesoporous bioactive glass (MBG), 1 mL of liquid PRF with 8 mg of mesoporous bioactive glass (5% Sr-MBG), 1 mL of liquid-PRF+PLLA-scaffold/siRNA-Sema4d, 0.5 mL of PIB-nanogel+0.5 mL of liquid PRF, 0.5 mL of PIB-nanogel+0.5 mL of liquid-PRF+osteoblasts (2×10⁶ cells).

The samples were fixed in 4% formaldehyde for 24 h at room temperature. A p-CT imaging system (p-CT50, Scanco Medical, Basersdorf, Switzerland) was used to evaluate new bone formation within the defect region. All samples were placed in a custom-made holder to ensure that the long axis of the drilled channel was oriented perpendicular to the axis of X-ray beam. All samples were calibrated by performing a scans with identical parameters at 55 kV and 114 mA with a thickness of 0.048 mm per slice in medium-resolution mode, 1024 reconstruction matrix, and 200-ms integration time. A Gaussian filter (sigma=0.8 and support=1) was used to remove noise. The mineralized bone tissue was differentially segmented to exclude the nonmineralized tissue with a fixed threshold (value=190). A series of slices starting at a distance of 1 mm proximal from the end of the growth plate with a length of 2 mm were chosen for the evaluation. For evaluation of bone regeneration within the defect, the central 2-mm-diameter region of the 2.5-mm-diameter defect was defined by drawing circular contour as area of measurement per slice, thus to obtain a consistent volume of interest (VOI) and to avoid including the native bone margins. All 3D images were generated by the built-in software of the p-CT.

### Example 9

### Use of Liquid-PRF as a Carrier for Regenerative Biomaterials in Collagen Synthesis of Skin Fibroblasts In Vitro

Normal skin tissues were harvested from a 12-year-old male donor undergoing cleft lip repair surgery. Collected tissues were washed three times with phosphate buffered saline (PBS; 150 mM NaCl, 20 mM sodium phosphate, pH 7.2) containing 1% antibiotics (100 U/ml penicillin G, 100 ug/ml streptomycin, HyClone, Thermo Fisher Scientific Inc) and fractionated into small pieces using sterilized surgical scissors. The skin tissue pieces were then transferred into T25 culture flasks with DMEM containing 20% fetal bovine serum (FBS; Gibco, Life Technologies Corporation) and 1% antibiotics in an incubator at 37° C. under 5% CO₂. On the 7th day, cells were seen around the skin tissue pieces. When cells reached confluency, cells were trypsinized and cultured in DMEM with 10% FBS. For all experiments, cells from passage 3 through 7 were used.

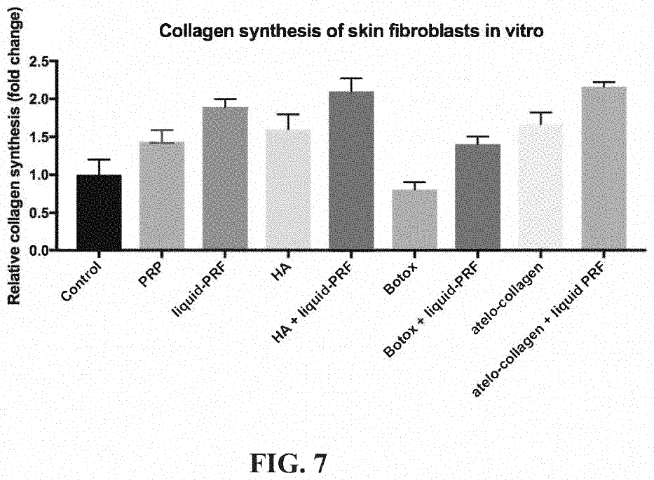

Platelet concentrates including PRP and i-PRF were incubated for 3 days at 37° C. in a 5% CO₂ atmosphere and thereafter conditioned media was harvested and utilized in future experiments as 20% of the total volume. Human skin fibroblasts were cultured in growth medium containing DMEM, 10% FBS and 1% antibiotics with/without 20% conditioned media from PRP or i-PRF at a density of 50,000 cells per well for collagen immunofluorescent staining in 24-well plates. Hyaluronic Acid was seeded in at 16 mg/mL. Similarly, Botulinum Toxin Type A (Botox) has a molecular weight of approximately 900 kDA, with a typical content of 100 units per vial (5 ng/vial of protein) (Carruthers et al. 2008). It was dissolved in a diluent (saline) and utilized at a concentration of 5 ng/mL. Atelo-collagen was utilized at a concentration of 4%. The medium was replaced twice a week when the experiments lasting longer than five days (FIG. 7).

Human skin fibroblasts were cultured in a 24-well plate at a density of 50,000 cells per well for 7 days. Cells were rinsed with PBS and fixed with 4% formaldehyde for 10 min. Cells were then permeabilized with 0.5% Triton X-100 (Merck, Germany) in PBS for 3 min at room temperature. Subsequently, cells were incubated with polyclonal rabbit to collagen type I (1:100, Boster Biological Technology Ltd, Wuhan, China) diluted in PBS containing 2% bovine serum albumin (BSA, Roche) for 1 hour, followed by incubation with FITC-conjugated-goat-anti-rabbit (1:200, Invitrogen) diluted in PBS for 1 hour. Finally, nuclei were stained by DAPI (blue fluorescence). After each step, the cells were washed with PBS three times. Images were taken using an Olympus DP71 fluorescence microscope (Olympus Co., Japan). Quantitative analysis of staining intensity was performed by Image J software.

### Example 10

### Use of Liquid-PRF as a Carrier for Regenerative Biomaterials in a Periodontitis Model

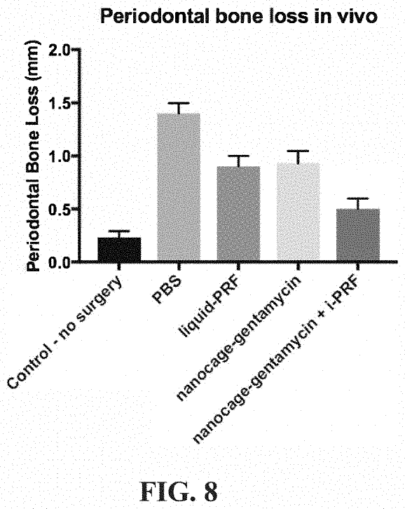

Eight-week-old female Sprague-Dawley rats (weighing from 180 to 200 g each) were purchased and used in this study. All surgical procedures used in this experiment were approved by the Ethics Committee for Animal Research, Wuhan University, China. Before starting, rats were divided into 3 groups (n=6): ligature with PBS as a negative control, ligature with pure PNA group, and ligature with GM@PNA-Ag group. Sterilized cotton thread ligatures (3/0) were placed around the cervix of the right and left second maxillary molars of rats anesthetized with pentobarbital sodium (50 mg kg⁻¹).^{[4]} Immediately after the surgical procedure, the negative control and experimental drug were applied locally to the second maxillary molars with an injection syringe, applied every other day. Then the rats received food and water ad libitum and were housed at a constant temperature of 22° C. The sagittal plane of each maxilla was placed in parallel to the x-ray beam axis. The scanning was performed at 70 kV and 114 μA and set at medium-resolution mode (15 μm per slice) as previously described.^{[5]} The vertical bone loss of the second maxillary molar was evaluated by measuring the distance between the cemento-enamel junction (CEJ) and the alveolar bone crest (ABC) at six points (lingual-mesial, lingual-middle, lingual-distal, buccal-mesial, buccal-middle, buccal-distal). The 3D regions of interest (ROIs) was defined as an area starting coronally by a 2D slice 15 μm below the root furcation with a height of 30 μm of the second maxillary molar. The results were presented as bone volume per total volume (BV/TV) (FIG. 8).

After fixation, the maxillas were decalcified in 10% EDTA, dehydrated, embedded in paraffin, sectioned to 4 μm thickness, and stained with hematoxylin and eosin (H&E) as in our previous studies.^{[5]} Vertical alveolar bone loss of the second maxillary molar were assessed for inflammation and alveolar bone destruction as assessed by measuring the length (mm) of the cemento-enamel junction (CEJ) to alveolar bone crest (ABC) between the second and third maxillary molars. To represent the macrophage polarization, immunohistochemical staining was performed to detect the expression of surface markers of macrophage. The sections were incubated with the primary antibody for CD206 and iNOS overnight at 4° C. In accordance with the manufacturer's protocol, the sections were incubated using the SP 9000 immunohistochemical kit (Zhongshan Biotechnology Co., Ltd, China) and visualized by 3,3-diaminobenzidine tetrahydrochloride (DAB) (Zhongshan Biotechnology Co., Ltd, China). And the cells positive for these markers were calculated.

In the preceding description, various aspects of claimed subject matter have been described. For purposes of explanation, specific numbers, percentages, components, ingredients and/or configurations were set forth to provide a thorough understanding of claimed subject matter. However, it should be apparent to one skilled in the art having the benefit of this disclosure that claimed subject matter may be practiced without the specific details. In other instances, features that would be understood by one of ordinary skill were omitted or simplified so as not to obscure claimed subject matter. While certain features and examples have been illustrated or described herein, many modifications, substitutions, changes or equivalents will now occur to those skilled in the art. It is, therefore, to be understood that the appended claims are intended to cover all such modifications or changes as fall within the true spirit of claimed subject matter.

1. A method of preparing an isolated serum fraction of liquid platelet-rich fibrin (PRF) comprising:
spinning a whole blood sample by centrifugation to separate erythrocytes;
separating a sample of liquid platelet-rich fibrin (PRF) from the whole blood sample without the addition of an additive;
immediately spinning the sample of liquid PRF by centrifugation carried out at 20 to 950 G for 2 to 8 minutes; and
prior to formation of a fibrin matrix of PRF, collecting an isolated serum fraction containing the liquid PRF using an application device.

2. The method of claim 1, wherein the centrifugation of the liquid PRF is carried out at 50 to 200 g.

3. The method of claim 1, wherein the application device is a needle, a syringe, or other application device configured to remove the isolated serum fraction of liquid PRF from the upper layer of a tube.

4. The method of claim 1, wherein the serum fraction comprises a platelet release from activated platelets.

5. The method of claim 4, wherein the serum fraction comprises at least one growth factor selected from the group consisting of PDGF, TGF-β1, VEGF, EGF and IGF.

6. A composition comprising an isolated serum fraction of liquid platelet-rich fibrin (PRF) and at least one regenerative biomaterial, the composition being obtained by a method comprising:
spinning a whole blood sample by centrifugation to separate erythrocytes;
separating a sample of liquid PRF from the whole blood sample without the addition of an additive;
immediately spinning the sample of liquid PRF by centrifugation carried out at 20 to 950 G for 2 to 8 minutes;
collecting the isolated serum fraction containing the liquid PRF using an application device; and
prior to formation of a fibrin matrix of PRF, combining the isolated serum fraction containing the liquid PRF with the regenerative biomaterial.

7. The composition of claim 6, wherein the serum fraction containing the liquid PRF and the regenerative biomaterial are combined in the application device.

8. The composition of claim 6, wherein the application device is a needle, a syringe, or other application device configured to remove the isolated serum fraction of liquid PRF from the upper layer of a tube.

9. The composition of claim 6, wherein in the method, the isolated serum fraction containing the liquid PRF is combined with the regenerative biomaterial immediately after collecting the isolated serum fraction containing the liquid PRF.

10. The composition of claim 6, wherein the regenerative biomaterial is a growth factor, an injectable material, a cell source, or a scaffold material.

11. The composition of claim 10, wherein the growth factor is selected from the group consisting of BMP1, BMP2, BMP3, BMP4, BMP5, BMP6, BMP7, BMP8, BMP9, BMP10, BMP11, BMP12, BMP13 BMP14, BMP15, BMP16, GDF1, GDF3, GD8, GDF9, GDF12, GDF14, PDGF, IGF, IGF-1, EGF, FGF2, FGF19, amelogenins, enamel matrix proteins, parathyroid hormones, transforming growth factor beta (TGFb1 and 2), epithelial, growth factor (EGF), vascular endothelial growth factor (VEGF) nerve growth factor (NGF), MMPs, TIMPs, caspase inhibitor, B-cell lymphoma 2 (BCL-2), human telemorase reverse transcriptase (hTERT), heat shock protein (HSP) 70, iNOS, IL-1Ra, sTNFR, siRNA, nitric oxide (NO), superoxide anion (02), IGF-1, IGF-2, bisphosphonates, LIF, COX-1, COX-2, PGE2, PGD2, fibronectin, tenascin, vitronectin, Vitamin E, Vitamin B12, chondroitin sulfates, androgens, thyroid hormones, Strontum, boron, zinc, magnesium, recombinant growth factors, or any combination thereof.

12. The composition of claim 11, wherein the recombinant growth factors are used for inhibiting inflammatory and catabolic pathways, stimulating anabolic pathways, or preventing cell senescence and apoptosis.

13. The composition of claim 10, wherein the injectable material is selected from the group consisting of scaffolds, hydrogels, microspheres, nanofibers, injectable gels, microcarriers, porous injectable particles, collagen, fibrin, silk, HA, alginate, agarose, agar, chitosan, gellan gum, polyethylene glycol (PEG) and its derivatives, poly(Lactide-co-glycolide) (PLGA), poly(l-lactic acid)(PLLA), polyethylene glycol diacrylate (PEGDA), deoxycholic acid, Botox, calcium hydroxylapatite (CaHA), polyacrylamide gel (PAAG), polyalkylimide gel (PAIG), polymethyl methacrylate (PMMA), silicone oil, AFT, injectable containing microparticles of PLLA, carboxymethylcellulose, nonpyrogenic mannitol, magnesium-carboxy-gluconate-hydrolactic gel, liquid injectable silicone, polycaprolactone Microspheres, Poly vinyl alcohol, DHT, dalteparin, and protamine microparticles, or any combination thereof.

14. The composition of claim 13, wherein the porous injectable particles are fabricated via solvent casting, freeze drying, gas foaming, salt leaching, rapid prototyping, or electro-spinning.

15. The composition of claim 10, wherein the scaffold material is selected from the group consisting of titanium, collagen, zirconium, sponges, graft materials, collagen membranes, collagen bi-products comprising synthetic or natural bone material, e-PTFE, d-PTFE, 3-D printing technologies, carbon-based nanomaterials such as carbon nanotubes (CNTs), titanium dioxide (TiO2) nanosheets, graphene, grapheme oxide, synthetic silicates and nanodiamonds (NDs), nHAp-poly(caprolactone) (nHAp-PCL), poly(ester urethane)-urea elastomer (PUEER/PUR), PEUUR nanofibers, synthetic bone grafts, allografts, xenografts, or any combination thereof.

16. The composition of claim 15, wherein the allografts and xenografts are selected from the group consisting of demineralized, freeze-dried bone allograft; allograft bone block, tissue; porcine bone grafts, bovine bone grafts, and mineralized bovine bone.

17. The composition of claim 10, wherein the cell source is selected from the group consisting of chondrocytes, osteoblasts, mesenchymal stem cells (MSCs), embryonic stem cells (ESCs), skin fibroblasts, fibroblasts mesenchymal stem cells (MSCs), adipocytes, adipose-derived stem cells, or embryonic stem cells (ESCs).

18. A method of treating an injured tissue in an individual comprising:
determining a site of tissue injury in the individual;
administering, using an injectable application device, an isolated serum fraction of liquid platelet-rich fibrin (PRF) into and around the site of tissue injury; and
administering, using an injectable application device, at least one regenerative biomaterial into and around the site of tissue injury.

19. The method of claim 18, wherein the liquid PRF was prepared by a method comprising:
spinning a whole blood sample by centrifugation to separate erythrocytes;
separating a sample of liquid PRF from the whole blood sample without the addition of an additive;
immediately spinning the sample of liquid PRF by centrifugation carried out at 20 to 950 G for 2 to 8 minutes; and
collecting the isolated serum fraction containing the liquid PRF using an application device.

20. The method of claim 19, wherein the regenerative biomaterial is administered into and around the site of tissue injury within 20 minutes of administering the serum fraction of liquid PRF into and around the site of tissue injury.

21. The method of claim 19, wherein the serum fraction of liquid PRF and the regenerative biomaterial are combined in the injectable application device to form a composition prior to administering the composition into and around the site of tissue injury.

22. The method of claim 18, wherein the tissue is connective tissue and the regenerative biomaterial is a growth factor, an injectable material, a cell source, or a scaffold material.
